(19) United States
(12) Patent Application Publication
(10) Pub. No.: US 2019/0282519 A1
(43) Pub. Date: Sep. 19, 2019

(54) PENCILLAMINE ORAL COMPOSITION

(71) Applicant: CRONUS RESEARCH LABS PRIVATE LIMITED, Hyderabad (IN)

(72) Inventor: Satya Srinivas Chetlapalli, Bangalore (IN)

(73) Assignee: CRONUS RESEARCH LABS PRIVATE LIMITED, Hyderabad (IN)

(21) Appl. No.: 16/357,006

(22) Filed: Mar. 18, 2019

(30) Foreign Application Priority Data
Mar. 19, 2018 (IN) 201841010052

(51) Int. Cl. A61K 31/145; A61K 9/48; A61P 13/02

(52) U.S. Cl. CPC A61K 31/145; A61P 13/02; A61K 9/4825

(57) ABSTRACT

The Invention relates to an oral pencillamine composition for reducing the dosing frequency in treatment of cysteinuria comprising of therapeutically effective amount of pencillamine and one or more pharmaceutically acceptable excipients.

### TECHNICAL FIELD OF THE INVENTION

The invention in general relates to pharmaceutical dosage forms. Particularly, the invention relates to oral composition of pencillamine and the method for preparing the same.

### BACKGROUND ART

Cystinuria, a rare genetic condition, is caused by mutations in the genes that encode the two subunits of the amino acid transport, resulting in failure of absorption of filtered dibasic amino acids including cystine in the proximal tubules. The very limited solubility of cystine in the physiological range of urine pH, leads to complicated and recurrent kidney stone formation leading to a higher risk of chronic kidney disease.

Sulfhydroxyl compounds which act as thiol-binding agents, such as D-penicillamine or tiopronin (α-mercaptopropionylglycine), are reported to reduce the formation of cystine crystals by reacting with cystine and generating more soluble mixed disulfide compounds (drug-cysteine complex).

Pencillamine is 3-mercapto-D-valine and is available as tablets and capsules of 250 mg strength. The usual dosage of pencillamine recommended in the treatment of cystinuria is 2 g/day for adults with a range of 1 to 4 g/day. Accordingly, patients have to take 16 capsules per day to meet dose of 4 g/day which would lead to poor patient compliance.

### SUMMARY OF THE INVENTION

Herein disclosed is pencillamine oral composition for reducing the dosing frequency and thereby improving the treatment compliance by the patients. The oral pencillamine composition as disclosed herein comprises of 500 mg of pencillamine and one or more pharmaceutically acceptable excipients. The oral pencillamine composition as described herein reduces the urinary cysteine of 500 mg/day by reducing the frequency of administration, which ultimately improves the patient compliance.

The oral pencillamine composition as described herein comprises of 500 mg of pencillamine, at least one filler and at least one lubricant.

The oral pencillamine composition is a hard gelatin capsule comprising of 500 mg of pencillamine, lactose and magnesium stearate, wherein the hard gelatin capsule is of 00 size.

### DETAILED DESCRIPTION OF THE INVENTION

The invention disclosed herein provides a pencillamine oral composition for reducing the dosing frequency and thereby improving the treatment compliance by the patients. The oral pencillamine composition as disclosed herein comprises of 500 mg of pencillamine and one or more pharmaceutically acceptable excipients. The oral pencillamine composition as described herein reduces the urinary cysteine of 500 mg/day by reducing the frequency of administration, which ultimately improves the patient compliance.

The pharmaceutically acceptable excipient is selected from the group comprising of fillers, lubricants or a combination thereof.

According to one embodiment the oral pencillamine composition comprises at least one filler and one lubricant. The filler is selected from the group consisting of directly compressible starches, hydrolysed starches, Lactose and its derivates dextrose, sorbitol, microcrystalline cellulose, dibasic calcium phosphate dehydrate, Calcium sulphate dehydrate or a combination thereof. The lubricant is selected from the group consisting of talc, stearic acid, magnesium stearate, calcium stearate, waxes, polyethylene glycol, surfactants or vegetable oil.

According to another embodiment the oral pencillamine composition is a hard gelatin capsule comprising of 500 mg of pencillamine, lactose and magnesium stearate, wherein the hard gelatin capsule is of 00 size.

### Example 1—Pencillamine Capsule Compositions

| | Name of the | Amount (in mg/unit) | | | |
|---|---|---|---|---|---|
| S. No. | Active/Excipient | F1 | F2 | F3 | F4 |
| 1 | Pencillamine | 500.00 | 500.00 | 500.00 | 500.00 |
| 2 | Lactose | 10.00 | 13.00 | 7.00 | 88.00 |
| 3 | Magnesium stearate | 5.00 | 7.00 | 7.00 | 12.26 |
| 4 | Isopropyl alcohol | q.s. | q.s. | q.s. | — |
| 5 | Water | — | — | — | q.s. |

Add Penicillamine & Lactose monohydrate into RMG and mix well for 5 minutes with impeller at slow speed. Slowly add granulating agent (Isopropyl alcohol or Water alcohol) in rapid mixer granulator over period of 60-90 seconds at impeller slow speed. Knead the wet mass for 60-90 seconds at impeller fast speed and chopper fast speed until get a good granular mass.

Unload the wet granular mass into FBD bowl with impeller slow speed. Air-dry the wet granular mass for 5 minutes to ensure proper fluidization. Dry the wet granular mass in fluid bed drier at an inlet temperature of 45° C. to get loss on drying of NMT 1% w/w.

Sift the dried granules through 30 ASTM sieve and collect the retentions and fines separately into in process container. Mill the retentions through the 1.5 mm or lmmmultimill screen with knives forward at slow speed & sift through 30 ASTM sieve.

Add magnesium stearate to the milled granules and blend it for 5 min. Fill lubricated blend in to Empty Hard gelatin size “1” capsules by using Automatic Filling capsule Machine.

We claim:

1. An oral pencillamine composition for reducing the dosing frequency in treatment of cysteinuria comprising of therapeutically effective amount of pencillamine and one or more pharmaceutically acceptable excipients, wherein the amount of pencillamine is 500 mg.

2. The oral pencillamine composition of claim 1 wherein the pharmaceutically acceptable excipient is selected from the group comprising of fillers, lubricants or a combination thereof.

3. The oral pencillamine composition of claim 1 wherein the filler is selected from the group consisting of directly compressible starches, hydrolysed starches, Lactose and its derivates dextrose, sorbitol, microcrystalline cellulose, dibasic calcium phosphate dehydrate, Calcium sulphate dehydrate or a combination thereof.

4. The oral pencillamine composition of claim 1 wherein the lubricant is selected from the group consisting of

5. The oral pencillamine composition of claim 1 is a hard gelatin capsule.
